(19) United States
(12) Patent Application Publication
(10) Pub. No.: US 2003/0084484 A1
(43) Pub. Date: May 1, 2003

(54) Commercial use of arabidopsis for production of human and animal therapeutic and diagnostic proteins

(76) Inventors: Newell Bascomb, Wayne, NJ (US); Mark Bossie, Robbinsville, NJ (US); Marina Skarjinskaia, Highland Park, NJ (US); Lynne Hirayama, Titusville, NJ (US); Gerald Hall, Morrisville, PA (US); Thomas Petty II, Ewing, NJ (US); Andrei Golovko, Westampton, NJ (US); Melissa Campo, Princeton Junction, NJ (US)

Correspondence Address: EDWARDS & ANGELL, LLP, P.O. BOX 9169, BOSTON, MA, 02209, US

(21) Appl. No.: 10/205,851

(22) Filed: Jul. 26, 2002

Related U.S. Application Data
(60) Provisional application No. 60/308,379, filed on Jul. 27, 2001.

(51) Int. Cl. A01H 1/00; C12P 21/02; C12N 5/04

(52) U.S. Cl. 800/288; 435/69.1; 435/419

(57) ABSTRACT

The invention provides methods that make it possible to take advantage of various growth parameters of Arabidopsis in order to grow dense populations of the plant in controlled indoor environments for the purpose of harvesting the biomass and isolating proteins, particularly recombinant proteins suitably for pharmaceutical applications.

### RELATED APPLICATION

This Application claims priority under 35 U.S.C. §119(e) to U.S. Provisional Application No. 60/308,379, filed Jul. 27, 2001, the entirety of which is incorporated by reference herein.

### FIELD OF THE INVENTION

This invention is related to the production of proteins in large-scale amounts using Arabidopsis thaliana.

### BACKGROUND OF THE INVENTION

Large-scale protein production is required to effectively exploit recombinant gene products, such as therapeutic proteins, for human use. While microbial systems often offer advantages up-front, in speed of cloning and producing transformed cells, there are often difficulties in the scale-up from laboratory to large fermentation vessels. Because many posttranslational processing steps are different in bacteria and eukaryotes there are certain categories of proteins that simply cannot be made in prokaryotic systems.

Mammalian and insect cell cultures have become widely used for the production of a variety of proteins, with probably the most significant advantage being post-translation processing. Otherwise, the media, equipment and fastidious culture conditions drive up production cost and are a distinct disadvantage to these systems. Yet another disadvantage of such systems is the potential for harboring virions or prions of concern to human health.

Transgenic animals have also been described for producing human proteins in milk, excreted in the urine or produced via eggs of avian species. Like animal cell culture, transgenic animals should provide proteins with the requisite post-translation modifications. However, transgenic animals are slow to produce, difficult to maintain, and not easily scaled-up. Production costs are fairly high and the same purification issues are a problem in these systems.

Using plants as a recombinant protein expression system or “bioreactor” is an attractive alternative to bacterial, yeast, insect, animal and cell-based production systems. There are many benefits to producing proteins in plants and the use of plants for the production of transgenic proteins is gaining widespread support.

Plant production systems allow for ease of purification free from animal pathogenic contaminants. Transformation methods exist for a large number of plant species. In the case of many seed plants and agricultural crops, the methods and infrastructure already exist for harvesting and handling large quantities of material. Scale-up is relatively straightforward and is based simply on production of seed and planting area. Thus, there is a substantial reduction in the cost of goods, reduced risks of mammalian viral or prion contamination, and relatively low capital requirements for raw material and production facilities as compared to producing similar material via mammalian cell culture or transgenic animals. Plants generally suffer only a single significant drawback and that is in the area of post-translational glycosylation of proteins. However, it has been demonstrated that in many cases the alternative carbohydrate modifications of plants do not cause deleterious effects or undesirable immunogenic properties to the glycoprotein.

A number of production systems have been developed for expressing proteins in plants. These include expressing protein on oil bodies (Rooijen et al., 109 Plant Physiology 1353-61 (1995); Liu et al., 3 Molecular Breeding 463-70 (1997)), through rhizosecretion (Borisjuk et al., 17 Nature Biotechnology 466-69 (1999)), in seed (Hood et al., 3 Molecular Breeding 291-306 (1997); Hood et al., In Chemicals via Higher Plant Bioengineering (ed. Shahidi et al.) Plenum Publishing Corp. 127-148 (1999); Kusnadi et al., 56 Biotechnology and Bioengineering 473-84 (1997); Kusnadi et al., 60 Biotechnology and Bioengineering 44-52 (1998); Kusnadi et al., 14 Biotechnology Progress 149-55 (1998); Witcher et al., 4 Molecular Breeding 301-12 (1998)), epitopes on the surface of a virus (Verch et al., 220 J. Immunological Methods 69-75 (1998); Brennan et al., 73 J. Virology 930-38 (1999); Brennan et al., 145 Microbiology 211-20 (1999)), and stable expression of proteins in potato tubers (Arakawa et al., 6 Transgenic Research 403-13 (1997); Arakawa et al., 16 Nature Biotechnology 292-97 (1998); Tacket et al., 4 Nature Medicine 607-09 (1998)). Recombinant proteins can also be targeted to the seeds, chloroplast or secreted to identify the location that gives the highest level of protein accumulation.

Most efforts to exploit plants in order to obtain biotechnological solutions to problems of protein production have focused on use of major row crops. The emphasis has largely been on biomass production and the agricultural industry has already developed the worlds greatest biomass production system, farming. During the last 7-8 years, there have been numerous examples of foreign proteins (e.g., vaccines, monoclonal antibodies, avidin and others) in crop plants. It has been clearly demonstrated that agricultural production plants can serve as very cost effective means of producing foreign proteins. In most cases, estimates of the cost of goods produced by such plants, in contrast to goods obtained from typical fermentation technology, indicates that using plants is on the order of 50-100-fold less expensive.

It is also true that production capability can be significantly higher for plant-based production systems when one accounts for all of the potential acres of land that could reasonably be planted for producing a specific product. However, as of today, such systems and approaches are not without significant flaws and disadvantages. As it pertains to production of highly regulated biologicals for pharmaceutical applications, one of the most severe drawbacks is the unregulated nature of outdoor production systems. It is either difficult to develop a validated and CGMP compliant process because of the variability of outdoor conditions or it can be a concern to grow these genetically modified organisms (GMOs) outdoors.

During the past decades, research in plant biotechnology has been largely driven by initial discoveries using a small and rapidly growing weedy plant, Arabidopsis thaliana (thale cress). This plant has many redeeming qualities for a role in the research laboratory. It is small, has a short life cycle, prolific seed generation capacity, has a relatively small and un-complex genome and is readily transformable by a variety of methods and there are many mutant varieties. During the past decade, the Arabidopsis genome has been completely sequenced marking the first higher plant species to reach that milestone. For these reasons, Arabidopsis became a common research organism for plant biotechnology. However, it has been widely recognized that this small weedy plant serves as only a model.

Thus, Arabidopsis has generally been exploited only in research settings. For those working in crop specific research programs, knowledge obtained from studies of Arabidopsis has typically been used to gain a fuller understanding of some of the world's major food crops and horticultural crops and to apply that understanding to modify and improve those species.

### SUMMARY OF THE INVENTION

Growth conditions, product manufacturing and regulatory needs are critical factors to consider when making biopharmaceutical or diagnostic materials. The methods according to the invention provide a highly reliable, rapid system that is scalable from the earliest testing and prototype stages up through full-scale production of recombinant proteins.

There are few plant species as amenable as Arabidopsis for the rapid generation of plants and seed. In one aspect, the invention provides a method of large-scale production of recombinant proteins from Arabidopsis by screening for genetic constructs and transgenic plants that express high yields of such proteins. Any suitable technique can be used, such as an Agrobacterium floral dip or vacuum infiltration transformation procedure. Preferably, the time from transformation to transgenic seed is less than 10 weeks, e.g., from about 8-10 weeks. In another aspect, a rapid transient expression analysis system is used, such as leaf and seedling infiltration or protoplast electroporation, to test proper function of new genetic constructs within days of making them.

Vectors used to introduce such recombinant constructs can include useful sequences, including, but not limited to: site-specific recombination sites to facilitate the specific integration into selected genomic loci, selectable markers to be used (e.g., BAR, NPTII, etc.) and/or other screenable markers such as GFP (green fluorescent protein or mutated or modified forms thereof), luciferase or GUS (betaglucuronidase). Preferably, a recombinant construct comprises a nucleic acid sequence encoding a protein of interest operably linked to a promoter and/or one or more genetic regulatory elements such as IRES (internal ribosome entry sites).

In one aspect, mutant recombinant proteins are screened for, either by random mutagenesis, or by rational design, or by a combination of such techniques, to identify constructs which proteins with desirable properties such as increased stability and/or activity. Recombinant constructs expressing such proteins are preferably tested in transient assays in parallel with constructs expressing wild-type forms of the protein.

Another way to generate variants for this type of biological “analog” testing is to change something in the production system that will affect a change in the final product. This can be readily accomplished in Arabidopsis by using a pre-existing Arabidopsis variety or by generating mutant varieties of the plants that alter the protein processing characteristics of the plant. Thus, any DNA information added to the system for making a new protein may be slightly altered depending on the host plant capabilities, to perform certain translational or post-translational modifications.

Glycosyslation is an example that is particularly relevant to this discussion. It is known that the sugars added to proteins during glycosylation differ between animals and plants. There is a core glycan that is largely the same but primarily differs by the addition of xylose and a-1-3 fucose and lack of terminal sialic acid. It is not yet certain which, if any and how much these differences will matter in terms of the efficacy and safety of plant-based products as pharmaceutical molecules. There is enough literature that suggests that such changes are inconsequential to the activity and safety and others, which suggest a down side to either one or both of these aspects. Having a suite of Arabidopsis mutants available to produce proteins having for instance altered glycan side-chain(s) is a distinct advantage to the systematic approach provided by the invention. For instance, small amounts of protein from wild-type and various mutant or engineered forms of Arabidopsis can be tested in parallel using in vitro functional assays to identify mutant or engineered forms of Arabidopsis for producing pharmaceutically acceptable recombinant protein products.

Additional examples of mutant lines that are useful include, but are not limited to, protease deficient strains and those mutants that have an increase in average biomass (particularly leafy biomass) in comparison with other lines of Arabidopsis. Here, the focus is to increase output, not necessarily to produce alternate forms of a product.

Thus, it is a preferred aspect of the invention, that before stable transgenic lines are made, determinations are made as to which constructs, which mutant forms, and which host system backgrounds, produce the most pharmacologically useful form of the desired protein.

One way of looking at a particularly preferred aspect of the present invention is that it begins where conventional work with Arabidopsis leaves off. In the past, Arabidopsis was used as a model to establish that certain proteins could be expressed in plants or to provide data regarding the characteristics of a particular expression vector. The protein and the vector would generally be commercially exploited in a different plant system. In contrast, the invention provides methods and systems for preselecting desired expression constructs and expressing that construct in Arabadopsis on a large scale, utilizing the optimal construct and Arabidopsis strain identified in pre-production assays, such as those described above. In one preferred aspect, the invention therefore comprises identifying a plant which produces an optimal amount and/or form of protein and producing large scale amounts of the protein in progeny of the plant, clonally related plants, or substantially, genetically identical plants.

Most preferably, the Arabidopsis strain selected and the expression system used is designed to maximize the protein yield per plant. This can include the use of multiple copies in a sequence in a gene, as well as expression vectors that are designed to result in the production of protein throughout as many portions of the plant as possible. These vectors are then introduced into Arabidopsis and expression induced while the plant is being grown under conditions designed to maximize the growth of plants and the expression of the protein. While optimized systems that maximize production are most preferred, suboptimal production that is economically viable is still considered within the scope of this aspect of the invention.

Generally, plants according to the invention are grown under conditions that favor production of leaf and root biomass even at the expense of diminishing the amount of seed or harvesting the plant prior to seed production and maturation.

In one aspect of the invention, Agrobacterium is used to introduce optimal vectors and constructs selected from the assays described above, for introduction into plant cells and for the growth and production of plants and/or seeds that stably express recombinant proteins of interest. Preferably, an infiltration method is used, such as a vacuum infiltration method.

Within a very short period of time and very small space it is possible to make hundreds of T1 transgenic lines that will, within a few weeks, give rise to thousands of each putative T1 transgenic line. From these thousands of putative transgenic T1 plants, screens are performed to assess which lines have the desired expression of the transgene. These lines are then allowed to self-pollinate giving rise to the T2 population in approximately eight weeks. Using standard Mendelian genetics as a guide, the T2 generation produced should consist nominally of 25% homozygous transgene lines for a single point of insertion. These lines can then be used to rapidly scale-up to production quantities of “pure-breeding” homozygous seed.

Desirably, plant growth occurs along a scale that is far in excess of that which would be used for research. For example, in a particular plant growth chamber such as a greenhouse or growth room, Arabidopsis may be the only plant being grown at any one time. However, it is unlikely that each plant in that greenhouse will contain the exact same construct with the exact same goal of maximizing production of the exact same protein or proteins. But growing the same plant, with the same expression system, designed to produce the same protein throughout that same greenhouse is likely using the present invention.

In a particularly preferred embodiment of the present invention, production continues on this scale over an extended period of time, of weeks, months and years. Thus, even if someone might consider growing a greenhouse full of Arabidopsis containing a single construct expressing a single protein for research purposes, it is unlikely that they would complete a life cycle of these plants only to begin a second, third, fourth and fifth planting under exactly the same conditions, for example, harvesting the complete area of the greenhouse and replanting the complete area of the greenhouse with the same type of plant expressing the same type of protein using the same kind of expression system, over and over again. Accordingly, one aspect of the present invention involves the production of a certain mass of protein per acre if grown in two dimensions or in cubic meters if grown in three dimensions such as stacked flats in a growth room.

In a particularly preferred embodiment in accordance with this aspect of the invention, production continues on this scale and/or for a period of at least six months so as to result in a production of a commercially meaningful amount of protein.

In one aspect, a growth room of about 20′×20′ (400 sq ft) is used to produce at least about 4 kg of total Arabidopsis biomass for harvesting in about 45-60 days when plants are grown on a single horizontal layer. In another aspect, plants are grown at more than one layer. For example, increasing to at least about six layers permits production of at least about 240 kg of plant biomass per room per growth period in about 45-60 days. Assuming between 6 and 8 growth/harvest cycles per year and assuming a modest expression of about 0.5% of total soluble protein, it is estimated that such a system would yield at least about 72 to 96 gm of purifiable protein of interest per year.

Accordingly, in one aspect, the invention comprises a method of producing a transgenic Arabidopsis strain under suitable conditions to achieve total plant biomass of at least about 10 kg and from that total plant biomass, reasonable quantities of purifiable engineered protein product can be obtained. Preferably, the method is scalable and can readily achieve greater levels of product by increasing the planted area, increasing the percent of total protein representing a desired protein, decreasing the amount of time necessary to achieve a certain biomass and percent desired protein or any combination of the above.

Particularly preferred embodiments of the present invention are methods of producing a desired protein from Arabidopsis. Proteins derived from these processes are also contemplated. These methods include the steps of providing a particular variety of Arabidopsis including at least one expression cassette, which will express at least one protein of interest. The protein can be heterologous or otherwise foreign to the plant.

### DETAILED DESCRIPTION

In contrast, in the current invention, the small weedy plant, Arabidopsis thaliana is used as a protein production host. The invention provides methods that make it possible to take advantage of various growth parameters of Arabidopsis in order to grow dense populations of the plant in controlled indoor environments for the purpose of harvesting the biomass and isolating proteins. In this regard, the invention provides methods of identifying parameters or inputs to maximize the amount of plant material grown per unit area or space, per unit time.

Definitions

The following definitions are provided for specific terms which are used in the following written description.

As used in the specification and claims, the singular form “a”, “an” and “the” include plural references unless the context clearly dictates otherwise. For example, the term “a cell” includes a plurality of cells, including mixtures thereof. The term “a protein” includes a plurality of proteins.

“Arabidopsis”, as used herein, refers to intact plants, or parts thereof. This term includes, without limitation, whole plants, plant cells, plant organs, plant seeds, protoplasts, callus, cell cultures, and any group of plant cells organized into structural and/or functional units. The use of this term in conjunction with, or in the absence of, any specific type to plant tissue as listed above or otherwise embraced by this definition is not intended to be exclusive of any other type of plant tissue.

“Plant cells” as used herein includes plant cells in plant tissue or plant tissue and plant cells and protoplasts in culture, or isolated or semi-isolated cells. “Plant tissue” includes differentiated and undifferentiated tissues of plants, including, but not limited to, roots, shoots, leaves, pollen, seeds, tumor tissue and various forms of cells in culture, such as single cells, protoplasts, embryos and callus tissue. The plant tissue may be in plant, or in organ, tissue or cell culture.

As used herein, “plant material” includes processed derivatives thereof, including, but not limited to: food products, food stuffs, food supplements, extracts, concentrates, pills, lozenges, chewable compositions, powders, formulas, syrups, candies, wafers, capsules and tablets.

“Screening” generally refers to identifying the cells exhibiting expression of a recombinant gene that has been transformed into the plant. Usually, screening is carried out to select successfully transformed seeds (i.e., transgenic seeds) for further cultivation and plant generation (i.e., for the production of transgenic plants). As mentioned below, in order to improve the ability to identify transformants, one may desire to employ a selectable or screenable marker gene as, or in addition to, the recombinant gene of interest. In this case, one would then generally assay the potentially transformed cells, seeds or plants by exposing the cells, seeds, plants, or seedlings to a selective agent or agents, or one would screen the cells, seeds, plants or tissues of the plants for the desired marker gene. For example, transgenic cells, seeds or plants may be screened under selective conditions, such as by growing the seeds or seedlings on media containing selective agents, such as antibiotics (e.g., hygromycin, kanamycin, paromomycin or BASTA®), the successfully transformed plants having been transformed with genes encoding resistance to such selective agents.

As used herein, a “multi-subunit protein” is a protein containing more than one separate polypeptide or protein chain associated with each other to form a single globular protein, where at least two of the separate polypeptides are encoded by different genes. In one preferred aspect, a multi-subunit protein comprises at least the immunologically active portion of an antibody and is thus capable of specifically combining with an antigen. For example, the multi-subunit protein can comprise the heavy and light chains of an antibody molecule or portions thereof. Multiple antigen combining portions can be encoded by different structural genes to generate multivalent antibodies.

In the case of a pharmaceutical product, the term “substantially pure”: generally refers to a product of at least 97% pure, more preferably at least 99% and even more preferably at least 99.99% pure.

By “interstitial fluid” is meant the extract obtained from all of the area of a plant not encompassed by the plasmalemma, i.e., the cell surface membrane. The term is meant to include all of the fluid, materials, area or space of a plant that is not intracellular (wherein intracellular is defined to be synonymous with innercellular) including molecules that may be released from the plasmalemma by this treatment without significant cell lysis. Synonyms for this term might be exoplasm or apoplasm or intercellular fluid or extracellular fluid.

The term “promoter” refers to the nucleotide sequences at the 5′ end of a structural gene which directs the initiation of transcription. Generally, promoter sequences are necessary, but not always sufficient, to drive the expression of a downstream gene. In the construction of heterologous promoter/structural gene combinations, the structural gene is placed under the regulatory control of a promoter such that the expression of the gene is controlled by promoter sequences. The promoter is positioned preferentially upstream to the structural gene and at a distance from the transcription start site that approximates the distance between the promoter and the gene it controls in its natural setting. As is known in the art, some variation in this distance can be tolerated without loss of promoter function. As used herein, the term “operatively linked” means that a promoter is connected to a coding region in such a way that the transcription of that coding region is controlled and regulated by that promoter. Means for operatively linking a promoter to a coding region are well known in the art.

A “recombinant gene” or “recombinant nucleic acid” is a gene/nucleic acid that is exogenous to, or not naturally found in, the plant to be transformed. Such foreign sequences include viral, prokaryotic, and eukaryotic sequences. Prokaryotic sequences include, but are not limited to, microbial sequences (e.g., for the production of antigens which may be administered as vaccines—viral sequences may also be used for this purpose). Eukaryotic sequences include mammalian sequences, but may also include sequences from non-mammals, even other plants. In one preferred aspect, a recombinant gene/nucleic acid encodes a human protein. A “recombinant gene” or “recombinant nucleic acid” may be naturally occurring, chemically synthesized, cDNA, mutated, or any combination of such sequences.

A “fusion protein” is a protein containing at least two different amino acid sequences linked in a polypeptide where the sequences were not natively expressed as a single protein.

As used herein, an “effector molecule” refers to an amino acid sequence such as a protein, polypeptide or peptide and can include, but is not limited to, regulatory factors, enzymes, antibodies, toxins, and the like. Non-limiting examples of desired effects produced by an effector molecule, include, inducing cell proliferation or cell death, to initiate an immune response or to act as a detection molecule for diagnostic purposes (e.g., the fusion may encode a fluorescent polypeptide such as GFP, EGFP, BFP, YFP, EBFP, and the like).

As used herein reduced glycosylation refers to at least 10% less glycosylation than levels observed in wild-type strains of Arabidopsis.

As used herein, “cultivated” or “cultivating” refers to growing Arabidopsis from seed until at least leaves are produced.

As used herein, “a diagnostic protein” or a “diagnostic reagent” refers to a protein or polypeptide whose reaction with a biomolecule is diagnostic of the presence of the biomolecule. As used herein, a “reaction with a biomolecule” refers to binding to, catalysis of, cleavage of, or modification of, the biomolecule. In one aspect, a diagnostic protein or reagent is directly or indirectly labeled, such that its reaction with the biomolecule produces a measurable response. An example of a diagnostic protein/reagent according to the invention is an antibody or an antigen binding fragment thereof. Antibodies may be double chain or single chain. If a double chain antibody, the chains of the antibody may be encoded on separate cistrons or as part of a polycistronic unit.

As used herein, an “effector molecule” refers to an amino acid sequence such as a protein, polypeptide or peptide and can include, but is not limited to, regulatory factors, enzymes, antibodies, toxins, and the like. Non-limiting examples of desired effects produced by an effector molecule, include, inducing cell proliferation or cell death, to initiate an immune response or to act as a detection molecule for diagnostic purposes (e.g., the fusion may encode a fluorescent polypeptide such as GFP, EGFP, BFP, YFP, EBFP, and the like).

As used herein, “biomass” refers to the total living tissue of Arabidopsis isolated from a particular area of a growing zone, i.e., a growth chamber. Preferably, such biomass is an amount of tissue excluding seed.

Arabidopsis Strains

Arabidopsis strains are commercially available and can be obtained, for example, from Lehle Seed (sales@arabidopsis.com) and various stock centers such as The Arabidopsis Biological Resource Center (ABRC) (The Ohio State University, 309 Botany & Zoology Bldg., 1735 Neil Avenue, Columbus, Ohio 43210 USA), Nottingham Arabidopsis Stock Centre (Plant Science Division, School of Biosciences, University of Nottingham, Sutton Bonnington Campus, Loughborough, LE12 5RD,UK). In one aspect, wild type Arabidopsis strains are used as the host background for the genetic constructs described below (see, e.g., http://www.arabidopsis.com/main/cat/seeds/wildtypes/!wl.html). Such strains can be used with or without markers to aid in the selection of transgenic lines.

Arabidopsis Mutants to Make Alternative Forms of Protein Products

As there are many mutant lines of Arabidopsis, it is also possible to attain and use lines that have defects in particular pathways that result in alternative forms of a protein being produced. As the Arabidopsis genome is completely sequenced, it is possible to identify, isolate, or create mutations in specific genes and pathways to achieve the desired effect. Examples of existing preferred mutants include the cgl and mur mutants that exhibit reduced levels of posttranslational glycosylation of proteins. Such strains can facilitate the production of certain type of proteins (i.e. human antibodies or human glycoproteins) by eliminating plant-specific protein glycosylation.

It is as yet unclear how significant a role glycosylation plays in the efficacy, safety and uses of plant-produced biologicals. There is a high degree of heterogeneity in the glycosylation patterns of endogenous plant glycoproteins as well as of recombinant proteins expressed in transgenic plants. This heterogeneity can be influenced by the growth stage of the plant as well as by specific growth conditions, such as temperature and light. Therefore, in one aspect of the invention, cgl, mur1 and mur4 mutant lines are used to create transgenic plants for production of proteins, particularly, where these may be used as therapeutic agents. In another aspect, genes that encode human glycosyltransferases are introduced into the background strain to produce a more human plant host system. See, e.g., as described in WO 0,034,490.

Other desired strains can be generated using standard mutagenesis techniques. In addition, mutagenized seeds are obtainable commercially, e.g., from Lehle Seed (http://www.arabidopsis.com/main/cat/seeds/M2/EMS/!2e.html).

Expression Cassettes

In preferred embodiments of the invention, wild-type or mutant or modified varieties Arabidopsis are engineered to express a gene of interest. Such a construct minimally comprises a nucleic acid sequence encoding a desired protein operably linked to a promoter and/or other regulatory elements to facilitate transcription of the gene and ultimately translation of the protein.

In one aspect, the gene construct is engineered, having in the 5′ to 3′ direction, a promoter, gene, and terminator. In another aspect, the gene construct comprises multiple coding regions linked on a common plasmid or co-transformed into the plants (such co-transformed constructs are collectively encompassed by the term “gene construct” as used herein). Multiple genes may be encoded as separate cistrons or as part of polycistronic units. In a further aspect, the gene construct comprises one or more IRES elements

Proteins

There is no preconceived limitation to the proteins to be produced by this invention, but there are certain categories of proteins which may be of particular relevance, given the need to produce certain products under regulated and reproducible conditions. In particular, this would include all classes of pharmaceutical and or diagnostic proteins for which Good Laboratory Practices and validated methods must be use during the course of production.

Proteins also may be expressed for their utility in nutraceuticals and cosmeceuticals, since these products are used for direct ingestion, injection or application (e.g., topical administration) to humans. Protein also may be expressed which are useful in the production of similarly regulated veterinarian products. However, generally, the methods and transgenic plants and plant cells described below are useful for any type of bulk protein production, whether regulated or not, and whether or not intended for human or animal consumption, or therapeutic or diagnostic uses.

Exemplary proteins which may be produced, include, but are not limited to: growth factors (e.g., such as Insulin-like Growth Factor I), receptors, ligands, signaling molecules; kinases, tumor suppressors, blood clotting proteins, cell cycle proteins, telomerases, metabolic proteins, neuronal proteins, cardiac proteins, proteins deficient in specific disease states, antibodies, antigens (e.g., such as oral antigens), proteins that provide resistance to diseases, antimicrobial proteins, Human Serum Albumin (e.g., human serum albumin), interferons, and cytokines.

Plants also may be transformed with one or more genes to reproduce enzymatic pathways for chemical synthesis or other industrial processes.

In another aspect, Arabidopsis is transformed with one or more genes to increase the utility of the plants as a source for large-scale protein production. Such genes include genes which make Arabidopsis resistant to diseases and insects, and/or genes which encode proteins providing antifungal, antibacterial or antiviral activity.

In one aspect, nucleic acid sequences are chosen encoding desired proteins wherein the nucleic acid sequences are designed to provide codons preferred by Arabidopsis. The characteristics of codon usage for Arabidopsis thaliana are described in Wada et al., “Codon Usage Tabulated From The GenBank Genetic Sequence Data,” Nucleic Acids Research 19 (Supp.) 1981-1986 (1991), for example.

As described further below, in one aspect, the invention provides a method for expressing a plurality of recombinant proteins. Such proteins may be expressed upon co-transformation of independent constructs or may be expressed from polycistronic expression units described further below. Such proteins can include those that in their native state require the coordinate expression of a plurality of structural genes in order to become biologically active. In one aspect, the protein requires the assembly of a plurality of subunits to become active. In another aspect, the protein is produced in immature form and requires processing, e.g., proteolytic cleavage, or modification (e.g., phosphorylation, glycosylation, ribosylation, acetylation, farnesylation, and the like) by one or more additional proteins to become active.

Non-limiting examples of such proteins include heterodimeric or heteromultimeric proteins, such as T Cell Receptors, MHC molecules, proteins of the immunoglobulin superfamily, nucleic acid binding proteins (e.g., replication factors, transcription factors, etc), enzymes, abzymes, receptors (particularly soluble receptors), growth factors, cell membrane proteins, differentiation factors, hemoglobin like proteins, multimeric kinases, and the like.

In preferred aspects of the invention, expression cassettes encode human proteins.

In one particularly preferred aspect, the expression cassette encodes one or more genes for monoclonal antibodies. Such genes can be obtained from murine, human or other animal sources. Alternatively, they can be synthetic, e.g., chimeric or modified forms of the genes encoding the heavy chain or light chain components of an antibody molecule. The order of the coding regions on the construct, e.g., heavy and light, or light then heavy, is not important. Genes coding for Heavy and Light polypeptides (e.g., such as variable heavy and variable light polypeptides) can be derived from cells producing IgA, IgD, IgE, IgG or IgM. Methods for preparing fragments of genomic DNA from which immunoglobulin variable region genes can be cloned are well known in the art. See, for example, Herrmann et al., Methods in Enzymol., 152:180-183 (1987); Frischauf, Methods in Enzymol., 152:183-190 (1987); Frischauf, Methods in Enzymol., 152:199-212 (1987). In one preferred embodiment, such as described below, such genes are encoded as part of polycistronic units.

Genes may also encode fusion proteins. For example, a structural gene may comprise a sequence encoding an effector polypeptide. As used herein, an “effector molecule” refers to an amino acid sequence such as a protein, polypeptide or peptide and can include, but is not limited to, regulatory factors, enzymes, antibodies, toxins, and the like. Non-limiting examples of desired effects produced by an effector molecule, include, inducing cell proliferation or cell death, to initiate an immune response or to act as a detection molecule for diagnostic purposes (e.g., the fusion may encode a fluorescent polypeptide such as GFP, EGFP, BFP, YFP, EBFP, and the like). In still another aspect, a protein may include an amino acid sequence which confers enhanced stability on a protein or which increases transcription of a protein. For example, a protein may be fused to a transcription activator capable of activating transcription from a promoter to which the gene is operably linked (see, e.g., Schwechheimer, et al., Funct. Integr. Genomics 1(1):35-43 (2000).

Regulatory Elements

Suitable regulatory elements for generating a particular construct will be selected based on the type of recombinant protein to be expressed. In general, the ability to express at high levels in all, or most, of the plant tissue of an Arabidopsis plant 20-40 days old is desired.

Plant Promoters

The gene constructs used may include all of the genetic material and such things as promoters, IRES elements, etc. These expression cassettes can either require some external stimuli to induce expression, such as the addition of a particular nutrient or agent, change in temperature, etc. or can be designed to express an encoded protein immediately and/or spontaneously during growth.

Thus, the expression of a gene encoding a desired protein may be controlled by constitutive or regulated promoters. Regulated promoters may be tissue-specific, developmentally regulated or otherwise inducible or repressible, provided that they are functional in the plant cell. Regulation may be based on temporal, spatial or developmental cues, environmentally signaled, or controllable by means of chemical inducers or repressors and such agents may be of natural or synthetic origin and the promoters may be of natural origin or engineered. Promoters also can be chimeric, i.e., derived using sequence elements from two or more different natural or synthetic promoters.

Preferably, a promoter used in the construct yields a high expression level of the gene, allowing for accumulation of the protein to be at least about 0.1-1%, at least about 1-5%, and more preferably, at least about 5% of total soluble protein, and/or yields at least about 0.1%, preferably at least about 0.5%, and most preferably, at least about 1%, of the total intercellular fluid (ICF) extractable protein.

The promoter should preferentially allow expression in all of the plant tissues, but most preferably, in all of the leaf, stem and root tissue. Additionally, or alternatively, the promoter allows expression in floral and/or seed tissue. In the present invention, the Arabidopsis Actin 2 promoter, the OCS(MAS) promoter and various forms thereof, the CaMV 35S, and figwort mosaic virus 34S promoter are preferred. However, other constitutive promoters can be used. For example, the ubiquitin promoter has been cloned from several species for use in transgenic plants (e.g., sunflower (Binet et al., Plant Science 79: 87-94 (1991); and maize (Christensen et al., Plant Molec. Biol. 12, 619-632 (1989)). Further useful promoters are the U2 and U5 snRNA promoters from maize (Brown et al., Nucleic Acids Res. 17, 8991 (1989)) and the promoter from alcohol dehydrogenase (Dennis et al., Nucleic Acids Res. 12, 3983 (1984)).

In another aspect, a regulated promoter is operably linked to the gene. Regulated promoters include, but are not limited to, promoters regulated by external influences (such as by application of an external agent, e.g., such as chemical, light, temperature, and the like), or promoters regulated by internal cues, such as regulated developmental changes in the plant. Regulated promoters are useful to induce high-level expression of a desired gene specifically at, or near, the time of harvest. This may be particularly useful in cases where the desired protein limits or otherwise constrains growth of the plant, or is in some manner, unstable.

Plant promoters which control the expression of transgenes in different plant tissues by methods are known to those skilled in the art (Gasser & Fraley, Science 244:1293-99 (1989)). The cauliflower mosaic virus ³⁵S promoter (CaMV) and enhanced derivatives of CaMv promoter (Odell et al., Nature, 3(13):810 (1985)), actin promoter (McElroy et al., Plant Cell 2:163-71 (1990)), AdhI promoter (Fromm et al., Bio/Technology 8:833-39 (1990), Kyozuka et al., Mol. Gen. Genet. 228:40-48 (1991)), ubiquitin promoters, the Figwort mosaic virus promoter, mannopine synthase promoter, nopaline synthase promoter and octopine synthase promoter and derivatives thereof are considered constitutive promoters. Regulated promoters are described as light inducible (e.g., small subunit of ribulose biphosphatecarboxylase promoters), heat shock promoters, nitrate and other chemically inducible promoters (see, for example, U.S. Pat. Nos. 5,364,780; 5,364,780; and 5,777,200).

Tissue specific promoters are used when there is reason to express a protein in a particular part of the plant. Leaf specific promoters may include the C4PPDK promoter preceded by the ³⁵S enhancer (Sheen, 15 EMBO, 12:3497-505 (1993)) or any other promoter that is specific for expression in the leaf. For expressing proteins in seed, the napin gene promoter (U.S. Pat. Nos. 5,420,034 and 5,608,152), the acetyl-CoA carboxylase promoter (U.S. Pat. Nos. 5,420,034 and 5,608,152), 2S albumin promoter, seed storage protein promoter, phaseolin promoter (Slightom et. al., Proc. Natl. Acad. Sci. USA 80:1897-1901 (1983)), oleosin promoter (Plant et al., Plant Mol. Bio. 25:193-205 (1994); Rowley et. al., 1997, Biochim. Biophys. Acta. 1345:1-4 (1997); U.S. Pat. No. 5,650,554; PCT WO 93/20216), zein promoter, glutelin promoter, starch synthase promoter, and starch branching enzyme promoter are all useful.

Generally, any plant expressible genetic construct is suitable for use in the methods of the invention. Particular promoters may be selected in consideration of the type of recombinant protein being expressed.

Other regulatory elements such as enhancer sequences also may be provided. For example, in one aspect, expression cassettes that contain multimerized transcriptional enhancers from the cauliflower mosaic virus (CaMV) 35S gene are used. See, e.g., Weigel, et al. Plant Physiol 122(4): 1003-13 (2000).

IRES Elements

It is generally accepted that the basic functional segment of DNA coding for a product includes a promoter followed by a protein-coding region and then a terminator. This basic, single cistronic (also termed “monocistonic”) format has long been the standard for expressing genes in any organism. According to the ribosome-scanning model, traditional for most eukaryotic mRNAs, the 40S ribosomal subunit binds to the 5′-cap and moves along the non-translated 5′-sequence until it reaches an AUG codon (Kozak Adv. Virus Res. 31:229-292 (1986); Kozak J. Mol. Biol. 108:229-241 (1989)). Although for the majority of eukaryotic mRNAs only the first open reading frame (ORF) is translationally active, there are different mechanisms by which mRNA may function polycistronically (Kozak Adv. Virus Res. 31:229-292 (1986)) such that a plurality of coding regions are expressed without each one being controlled by a separate promoter.

Accordingly, in one aspect of the invention, expression cassettes are provided which are translationally regulated using IRES technology. Thus, the present invention is not limited to gene constructs which rely on the use of promoters for each coding region.

The IRES element may be one of those previously described (Atebekov et al. WO 98/54342), or an artificial IRES, active in plant cells. For multi-IRES containing constructs, it may be useful to use IRES elements having different DNA sequences. Recently a new tobamovirus, crTMV, has been isolated from Oleracia officinalis L. plants and the crTMV genome has been sequenced (6312 nucleotides) (Dorokhov et al., 332 Doklady of Russian Academy of Sciences 518-22 (1993); Dorokhov et al., 350 FEBS Lett. 5-8 (1994)).

Unlike the RNA of typical tobamoviruses, translation of the 3′-proximal CP gene of crTMV RNA occurs in vitro and in planta by a mechanism of internal ribosome entry which is mediated by a specific sequence element, IRES_{CP} (Ivanov et al. Virology 232, 32-43 (1997)). The results indicated that the 148-nucleotide region upstream of the CP gene of crTMV RNA contained IRES_{CP} promoting internal initiation of translation in vitro and in vivo (protoplasts and transgenic plants).

Recently it has been shown (Skulachev et al., Virology 263:139-154 (1999)) that the genomic RNAs of tobamoviruses contain a sequence upstream of the MP gene that is able to promote expression of the 3′-proximal genes from chimeric mRNAs operably linked to the sequence in a cap-independent manner in vitro. The 228-nucleotide sequence upstream from the MP gene of crTMV RNA (IRES_{MP228}^{CR}) mediates translation of the 3′-proximal GUS gene from bicistronic transcripts. A 75-nucleotide region upstream of the MP gene of crTMV RNA is still as efficient as the 228-nucleotide sequence. Therefore, the 75-nucleotide sequence contains an IRES_{MP} element (IRES_{MP75}^{CR}). It has been found that in similarity to crTMV RNA, the 75-nucleotide sequence upstream of genomic RNA of a type member of tobamovirus group (TMV UI) also contains IRES_{MP75}^{UI} element capable of mediating cap-independent translation of 3′-proximal genes.

The tobamoviruses provides a new example of internal initiation of translation, which is markedly distinct from IRES's shown for picomaviruses and other viral and eukaryotic mRNAs. The IRES_{MP} element capable of mediating cap-independent translation is contained not only in crTMV RNA but also in the genome of a type member of tobamovirus group, TMV UI, and another tobamovirus, cucumber green mottle mosaic virus. Consequently, different members of tobamovirus group contain IRES_{MP}.

The present invention thus also includes production of proteins based on expression of polycistronic gene constructs using any combination of IRESes and/or promoters.

By way of example, two specific IRES elements are used in demonstration of this invention. Nucleotide sequence of two IRESes from the genome of the crucifer tobacco mosaic virus (crTMV):

IRESmp75^{cr}:

| | |
|---|---|
| 5′TTCGTTTGCTTTTTGTAGTATAATTAAATATTTG | (SEQ ID NO. 1) |
| TCAGATAAGAGATTG | TTTAGAGATTT GTTCTTTG |
| TTTGATA3′ | |

IREScp148^{cr}:

| | |
|---|---|
| 5′GAATTCGTCGATTCGGTTGCAGCATTTAAAGCGG | (SEQ ID NO. 2.) |
| TTGACAACTTTAAAAGAAGGAAAAAGAAGGTTGAAG | |
| AAAAGGGTGTAGTAAGTAAGTATAAGTACAGACCGG | |
| AGAAGTACGCCGGTCCTGATTCGTTTAATTTGAAAG | |
| AAGAAA3′ | |

Accordingly, one aspect of the present invention is directed to a recombinant nucleic acid molecule containing from 5′ to 3′, a transcription initiator and a plurality of structural genes, each separated by an internal ribosome binding sequence (IRES).

Constructs comprising IRES elements are described further in PCT/US02/17927, filed Jun. 7, 2002, the entirety of which is incorporated by reference herein.

Targeting Sequences

In preferred embodiments, expression products are targeted to a specific location in a plant cell, such as the cell membrane, extracellular space or a cell organelle, e.g., a plastid, such as a chloroplast. In a preferred embodiment, expression products are targeted to the extracellular space, thus enabling purification based on the isolation of the intracellular fluids. See, for example, U.S. Pat. Nos. 6,096,546, 6,284,875, and WO 0,009,725.

Proteins can be targeted to specific sub-cellular or extracellular locations by virtue of targeting sequences. In some cases the sequence of amino acids is synthesized as the amino terminal portion of the polypeptide and is cleaved by proteases, after, or during, the translocation or localization process. For instance, the model of the protein secretion pathway in eukaryotes is that following ribosome binding to mRNA and initiation of translation the nascent polypeptide chain emerges. If it is a protein destined for secretion, the emerging amino terminus of the protein is recognized by signal recognition particle (SRP) that brings about a temporary stalling of translation while an mRNA, ribosome and SRP complex docks with the endoplasmic reticulum (ER). After docking, translation resumes, although now the polypeptide chain is co-translationally translocated through to the ER lumen.

It is possible for proteins to be translocated post-translationally; however, this process in vivo is far less efficient and generally is not considered the normal route of entry into the ER. The signal sequences for targeting proteins to the endomembrane system for localization in the vacuole or for secretion are similar in plants and animals. Signaling peptides may be adapted for use in the present invention (e.g., prepared with suitable ends for cloning in-frame with any other gene) in accordance with standard techniques.

In one aspect, a expression cassette encoding a desired protein comprises a signal sequence fused in frame to sequences encoding the desired protein. In one preferred aspect, the signal sequence is one which can direct the expression product of the gene to a secretory pathway.

As antibodies are normally secreted proteins—the secretion process plays an important role in the production of the mature antibody molecules. To accomplish this in plants, the genes are synthesized (e.g., cloned) having either their native mammalian signal peptide encoding region, or as a fusion in which a plant secretion signal peptide is substituted. The fusion between the signal peptide and the protein should be such that upon processing by the plant, the resultant amino terminus of the protein is identical to that which is generated in the human host.

In a preferred embodiment, the secretion targeting signal from the calreticulin protein is used. It has been demonstrated that this plant signal peptide is efficient at targeting foreign proteins to the apoplastic space of the plant (see, e.g., Borisjuk et al., 17 Nature Biotechnology 466-69 (1999)). Other plant protein signal peptides may also be used such as those described for barley (α-amylase, During et al. 15 Plant Molecular Biology 287-93 (1990); Schillberg et al. 8 Transgenic Research 255-63 (1999)).

Targeting proteins to the endomembrane system of a plant is a preferred embodiment of the present invention for those proteins that normally require amino-terminal processing to achieve their mature form, because it provides for the proper maturation of the amino terminus of the protein. Further, localization to specific regions of the endomembrane system can be accomplished if the protein of interest either has, or is, engineered to contain additional targeting 3information (see, e.g., as described in: Voss et al., 1 Mol. Breeding 39-50 (1995); During et al., 15 Plant Mol. Biol. 281-93 (1990); Baum et al., 9 Mol. Plant-Microbe Interact. 382-87 (1996); DeWilde et al., 114 Plant Sci. 231-41 (1996); Ma et al., 24 Eur. J. Immunology 131-38 (1994); Schouten et al., 30 Plant Mol. Biol. 781-93 (1996); Firek et al., 23 Plant Mol. Biol. 861-70 (1993); Artsaenko et al., 8 Plant J. 745-50 (1995); Conrad & Fiedler 38 Plant Mol. Biol. 101-09 (1998)).

Targeting to organelles such as plastids (e.g., chloroplast and mitochondria) is also advantageous for achieving the desired amino-terminal maturation because targeting to either of these locations is dictated by an amino-terminal signal sequence that subsequently undergoes a cleavage event. In preferred embodiments, the signaling peptides direct the expression products to a plastid (e.g., a chloroplast) or other subcellular organelle. An example is the transit peptide of the small subunit of the alfalfa ribulose-biphosphate carboxylase (Khoudi et al., 197 Gene 343-5 (1997)). A peroxisomal targeting sequence refers to any peptide sequence, either N-terminal, internal, or C-terminal, that can target a protein to the peroxisomes, such as the plant C-terminal targeting tripeptide SKL (Banjoko et al., 107 Plant Physiol. 1201-08 (1995)).

On the other hand, nuclear localization signals are not naturally restricted to the 5′ end position (amino terminus) of a protein and are not proteolytically removed by any known cellular mechanisms. Thus, from a processing stand-point targeting proteins to the nucleus may not be as desirable.

Additionally, or as an alternative to targeting proteins to specific subcellular locations, in one aspect, “epitope tags” and/or site specific cleavage sites are added to create a fusion protein. The utility of such tags is that they can provide a convenient purification mechanism. For instance, a small peptide comprising the critical amino acid sequence from biotin for binding to streptavidin can be engineered on to the 5′ end of a gene of interest. The newly synthesized protein can then be captured by many known methods fundamentally based on biotin:straptavidin binding. If it is desirable to remove the “biotin-like” peptide from the protein, it is possible to also include a protease recognition site. The protease recognition site can be inserted downstream from the “epitope tag” sequence and just before the sequence encoding the mature form of the desired protein. Those skilled in the art will recognize that there are numerous choices for epitope tags and proteases (such as factor Xa, Tobacco Etch Virus protease, enterokinase, etc.) and that the choice of the preferred site and protease may depend on the specific protein amino acid and DNA sequence in question.

As described above, the selection of regulatory elements, such as promoters, enhancers, IRES elements, and signal sequences will generally depend on the type of protein being expressed. For example, In one aspect, some preferred constructs for the purpose of making an IgG would include constructs having 5′ Arabidopsis Actin 2 promoter: calreticulin (any plant) signal peptide: coding region for the mature portion of the IgG heavy chain gene: translational stop signals: IRES (mp75 cp148): BAR: transcriptional stop and polyadenylation sequence and a second construct containing similar elements as above, replacing the heavy chain gene with the light chain gene, and replacing the BAR gene with an alternative selection/screening marker such as GFP. Alternatively, in another preferred embodiment, the heavy chain and light chain genes are on the same DNA construct.

Vectors

In general, suitable expression vectors could be any vector system known to be useful in transforming plants. In general, such a vector would contain one or more sequences for stably replicating the vector in a plant cell, either episomally, or as part of an endogenous plant chromosome. Sequences for facilitating integration into a plant chromosome may be provided. In some aspects, it is desired to provide origins of replication from different types of cells to facilitate amplification in one type of cell and protein expression in another. For example, while generally, protein expression will be obtained in a plant cell, amplification may be performed in a prokaryotic cell (e.g., bacterial cell) to obtain suitable quantities of nucleic acid for subsequent transformation of a plant cell.

In the spirit of the current invention, there is no particular distinction made with regards to the exact nature of the genetic construct to be introduced into Arabidopsis plants meaning, that any nucleic acid (DNA or RNA construct) that is expressible in Arabidopsis is suitable under this invention including viral-based expression systems. However, as one aspect of this invention relates to the advantages of the speed at which new genes can be transformed into Arabidopsis and produce significant amounts of seed in succeeding generations, the Agrobacterium floral dip and vacuum infiltration method are preferred methods to introduce genes for stable integration into the genome and therefore, constructs suitable for such techniques are especially preferred.

For example, for Agrobacterium-mediated transformation, one preferred vector is a Ti-plasmid derived vector. Other appropriate vectors that can be used are known in the art. Suitable vectors for transforming plant tissue and protoplasts have been described by deFramond, A. et al., Bio/Technology 1, 263 (1983); An, G. et al., EMBO J. 4, 277 (1985); and Rothstein, S. J. et al., Gene 53, 153 (1987).

Other sequences for facilitating site-specific genome integration and/or controlled excision and/or reinsertion into the genome may also be provided. For example, the Cre/lox system can be used to obtain targeted integration of an Agrobacterium T-DNA at a lox site in the genome of Arabidopsis. Site-specific recombinants, and not random events, are preferentially selected by activation of a silent lox-neomycin phosphotransferase (nptII) target gene. Cre recombinase can be provided transiently by using a co-transformation approach. See, e.g., as described in Vergunst, et al., Plant Mol Biol 38(3): 393-406 (1998).

A vector suitable for chloroplast transformation is used. Chloroplasts are prokaryotic compartments inside eukaryotic cells. Since the transcriptional and translational machinery of the chloroplast is similar to E. coli (Brixey et al., 1997), it is possible to express prokaryotic genes at very high levels in plant chloroplasts than in the nucleus. In addition, plant cells contain up to 50,000 copies of the circular plastid genome (Bendich 1987) which may amplify a recombinant gene like a plasmid, enhancing levels of expression. Chloroplast expression may be a hundred-fold higher than nuclear expression in transgenic plants (Daniell, WO 99/10513).

Therefore, in one aspect, the expression cassette is cloned into a chloroplast vector. Preferably, the expression cassette comprises a recombinant gene operably linked to a chloroplast promoter (e.g., such as the 16S rRNA promoter). In one aspect, a selectable marker gene (e.g., such as aminoglycoside adenyl transferase (aadA), conferring resistance to spectinomycin). A terminator downstream of the recombinant gene and/or the selectable marker gene may be provided (e.g., such as the terminator sequence from the psbA 3′ region (the terminator from a gene coding for photosystem II reaction center components) from the Arabidopsis chloroplast genome. Preferably, the vector additionally encodes Arabidopsis chloroplast genome as flanking sequences for homologous recombination.

Selectable Markers and/or Reporter Genes

Selectable markers, such as antibiotic (e.g., kanamycin and hygromycin, nptII, hpt) resistance, herbicide (glufosinate, imidazlinone, glyphosate, AHAS, EPSPS) resistance or physiological markers (visible or biochemical) are used to select cells transformed with the nucleic acid construct. Non-transgenic cells (i.e., non-trans formants) on the other hand, are either killed or preferentially do not grow under the selective conditions. In one aspect, a selectable marker gene is a gene which encodes a protein providing resistance or physiological markers. However, in another aspect, a selectable marker gene is a gene encoding an antisense nucleic acid.

Reporter genes may be included in the construct or they may be contained in the vector that ultimately transports the construct into the plant cell. As used herein, a “reporter gene” is any gene which can provide a cell in which it is expressed with an observable or measurable phenotype.

Expression of reporter genes yields a detectable result, e.g., a visual colorimetric, fluorescent, luminescent or biochemically assayable product; a selectable marker, allowing for selection of transformants based on physiology and growth differential; or display a visual physiologic or biochemical trait. Commonly used reporter genes include lacZ (β-galactosidase), GUS (β-glucuronidase), GFP (green fluorescent protein and mutated or modified forms thereof), luciferase, or CAT (chloramphenicol acetyltransferase), which are easily visualized or assayable. Such genes may be used in combination or instead of selectable markers to enable one to easily pick out clones of interest. In one aspect, a selectable marker gene is a gene encoding a protein product.

Selectable markers can also include molecules that facilitate isolation of cells which express the markers. For example, a selectable marker can encode an antigen which can be recognized by an antibody and used to isolate a transformed cell by affinity-based purification techniques or by flow cytometry. Reporter genes also may comprise sequences which are detected by virtue of being foreign to a plant cell (e.g., detectable by PCR, for example). In this embodiment, the reporter need not express a protein or cause a visible change in phenotype.

Transformation of Arabidopsis

Methods for transferring and integrating a DNA molecule into the plant host genome are well known. Methods such as Arabidopsis vacuum-infiltration or dipping are preferred because many plants can be transformed in a small space, yielding a large amount of seed to screen for transformants. Agrobacterium typically transfers a linear DNA fragment (T-DNA) with defined ends (T-DNA borders) making it a preferred method as well. Direct DNA transformation, such as microinjection, chemical treatment, or microprojectile bombardment or biolistics (preferred for chloroplast mediated transformation) are also useful. Barring any limitations on the size of the recombinant construct, gene encoding sequences could be delivered into plants using viral vectors. The plant cells transformed may be in the form of protoplasts, cell culture, callus tissue, suspension culture, leaf, pollen or meristem. As a first stage, expression need only be transient, i.e., for a period of time to establish the suitability of the construct being used to generate subsequent stable transformed lines. Rapid transformation systems include, but are not limited to, floral dip or vacuum infiltration (Bechtold, et al., C.R. Acad. Sci. Paris, 316 Life Sciences 1194-99 (1993)); leaf and seedling infiltration (Kapila, et al., 122 Plant Science 101-108 (1997)), and protoplast electroporation.

In one preferred embodiment, Arabidopsis plants of an appropriate genotype are grown until they are flowering. Transformation of Arabidopsis is most conveniently performed by dipping developing floral tissues into an Agrobacterium solution. This step can be done with or without subjecting the small plants (35 days old or so) to a vacuum during the dipping stage. Within weeks of the floral dip, the Arabidopsis plants set seed that can be harvested and screened for those T1 plants that contain a gene of interest. See, e.g., Clough and Bent, Plant J. 16: 735-43 (1998).

In a preferred embodiment, this is accomplished by spreading the seed at a density of approximately 10 or greater seeds per square foot on a potting soil mixture (e.g., Metromix 350) and then applying a spray application of glufosinate or phophinothricin at rates sufficient to kill untransformed plants. The T1 transgenic plants expressing the selectable marker (BAR in this example) survive this treatment and are readily identifiable within 1-3 days after application of the selection agent. There are other methods and selectable agents that can be used, and are encompassed within the scope of the invention, but this method is preferred because of the simplicity and high throughput capabilities.

Identifying Optimal Constructs

The T1 plants are grown to maturity, allowing them to self-pollinate. In a preferred embodiment, a transient expression assay is performed in order to identify a genetic construct that is optimal for a particular protein production scheme contemplated. More preferably, a series of constructs are introduced in parallel to screen for constructs which exhibit suitable properties of protein expression, protein modification, protein stability and/or activity. At least one construct will express a wild-type protein, while one or more other constructs express randomly mutagenized and/or rationally mutagenized proteins.

Expression of such constructs is evaluated using an assay of suitable sensitivity for the protein of interest and a small amount of tissue can be tested from each surviving transformed T1 plant to confirm the expression/activity of the desired product. Such a test can be used to identify plants expressing a desired protein at the highest relative amounts and/or which express proteins having particular desired activities or levels of activities. In one preferred aspect, at least about 50, at least about 100, at least about 250, or at least about 500, constructs are tested in parallel.

In another aspect, a small amount of plant tissue or interstitial fluid is removed (e.g., large enough to obtain a suitable protein sample) and the tissue/interstitial fluid is crushed or captured by vacuum infiltration and subjected to an appropriate assay for measuring protein levels and/or activity. Any suitable assay for evaluating protein levels/activity may be selected. In one aspect, the assay is an immunoassay.

For example, the sample can be centrifuged and blotted on a suitable type of membrane filter (e.g., PVDF) to bind proteins. Preferably, the membrane is washed and then incubated in the presence of primary and secondary antibodies. The primary antibodies recognize and bind to the protein of interest and the secondary antibody binds to the primary antibody. The secondary antibodies are typically linked to either Alkaline Phosphatase or Horse Radish Peroxidase enzymes, permitting detection to be made by addition of a simple coloro- or fluormetric substrate. Similarly, an ELISA assay performed in multi-well plates can be used for detection of one or more protein(s) of interest. Such methods are generally known to those skilled in the art and may be modified as required to suit the detection of any specific protein.

To additionally, or alternatively, confirm the presence of the expression cassettes or “transgene(s)” in Arabidopsis, a variety of assays may be performed. Such assays include, for example, molecular biological assays, such as Southern and Northern blotting and PCR; biochemical assays, enzymatic function assays; electrophoretic assays; chromatographic assays; by mass spectrometry; by plant part assays, such as leaf or root assays; and also, by analyzing the phenotype of the whole regenerated plant.

The T2 and T3 generation seed can be similarly screened to identify plant lines with the highest level of production and most stable genetic constructs. In general, it is preferred to obtain plant lines that are homozygous for the gene(s) inserted and this is generally accomplished and confirmed by obtaining second and third generations. This is based on the fundamental principles of Mendelian genetics. If more than one gene is to be inserted and the genes are not physically linked together, it may take more generations to screen for a line that is homozygous at each locus. In any case, Arabidopsis provides a particular advantage over typical crop species because of the ease and speed of producing the progeny. It takes only 8-10 weeks to complete a generation cycle in Arabidopsis. Each single plant can be expected to produce at least 200 progeny seeds and more often it is significantly more than this (e.g., about 500 seeds).

Thus, in one aspect, the process is hierarchical, screening first T1 generations to identify constructs with desired properties and then selecting optimal T1 plants expressing such constructs, to generate optimal subsequent generations of plants with stable “predetermined expression properties,” i.e., stable transgenic lines. Transient assays may also be performed in a hierarchical manner, i.e., screening constructs first in cell-based assays and then screening optimal constructs identified in the first assay in T1 generations. In one particularly preferred embodiment, plants are screened to identify plants which express the highest amount of protein for a given amount of biomass. In one aspect, a plant line is identified which produces at least about 50, at least about 100, at least about 150, at least about 200 grams of biomass per square feet of plant cultivated.

Large Scale Production of Proteins

In one aspect, a variety of Arabidopsis containing at least one gene construct is grown under conditions that will promote the production of vegetative and leafy biomass. In short, this means healthy plants with a robust leaf system and harvested prior to the production of mature seed. For the purpose of scale-up, a certain population of the stable transgenic plants are grown under favorable conditions for producing seed in order to obtain at least about 200 seed from each individual plant. The Arabidopsis (seed or mature plant) is then harvested and one or more proteins of interest are isolated from the harvested plants. Where multiple recombinant proteins are produced, these may be produced as separate proteins or a multi-subunit complexes. Preferably, such multi-subunit complexes are functional as assembled.

The Arabidopsis strain used for large-scale production according to the invention, expresses known quantities of protein with known levels/types of activity and with known modification patterns. Similarly, the biological traits of the plant itself are known (e.g., particularly its affect on protein stability, targeting, modification, etc.). Thus, in contrast to methods of using Arabidopsis in the prior art, for large scale protein production, a preset, preselected Arabidopsis and expression system are provided with “predetermined expression properties.” This means that through the transient expression assays described previously, the nature of the protein expressed, the degree of expression, the point of expression within the plant or plant cells (leaf, root, whole plant, apoplast, ER, chloroplast), the preferred conditions, the preferred expression vector, the yield, etc., have already been determined.

For example, for a particular strain of Arabidopsis being grown on a large scale, it is known that this variety of Arabidopsis will express a roughly predictable amount of a foreign/heterologous protein if harvested on a certain day after planting and when grown under specific conditions. Plants or seeds having predetermined expression properties are provided for large-scale growth of Arabidopsis for the production of biomass of at least one intended protein.

This distinction will be best illustrated by a discussion of growth relative to such factors as time, area, yield and conditions. However, since time and area, for example, are scalable, it is best to pick one set of conditions as being illustrative and not limiting. Consider therefore, a plant growth chamber or growing room of 20 feet×20 feet containing a single layer of plant growth medium (natural soils, commercial and artificial soils, hydroponic mediums). The term “plant growth chamber” in accordance with the present invention includes any type of space which can be completely isolated from natural light, water, etc., or can be a greenhouse that can allow for a variable amount of exposure to natural sunlight, rain, etc. The term can also encompass a 20′×20′ area of an exposed or covered field such as those used in hydroponics or conventional soil-based farming.

In one aspect, Arabidopsis is grown under conditions that promote the production of a vegetative and leafy biomass. Preferably, plants are generally exposed to between about 8-10 hours of sunlight or suitable growth light conditions and maintained at a temperature of between about 18° C. to about 24° C. The growth medium will be supplied with sufficient nutrients (fertilizer) to promote vigorous growth (for example, Miracle Grow brand plant food or other similar product). In the case of soil growth, this is best performed by bottom watering to maintain a moist, but not overly saturated soil throughout the growth period.

In accordance with one aspect of the present invention, a plant growth chamber is be planted with a single variety of Arabidopsis, including at least one expression cassette, which will express at least one protein of interest under the conditions described above. Indeed, the combination of plant variety and cassette will have already been tested and characterized such that the protein expressed is known, and the degree of expression is known to a reasonable approximation, so that yield can be estimated based on the harvesting of a certain amount of Arabidopsis per chamber.

Ideally, plants being grown under suitably defined conditions are harvested between about 30 and 80 days, more preferably 40-70 days, and most preferably between about 45-60 days after planting. The most preferred number of days to harvest is generally predefined in the earlier stages which defined the most suitable host variety of Arabidopsis, the most preferred expression cassette and the best biomass-to-protein yield for the desired protein. In general, the target date for harvest is determined to be at or around the time of raceme emergence and up to and around the time just prior to the formation of seed. This time window is targeted because this permits the amount of harvestable leafy and root biomass to be maximized.

Although further growth can result in still more production of plant biomass, these tissues (stalk, flowers, seed pods and seed) generally are not the intended target tissue for the purpose of commercial large-scale protein production from Arabidopsis. Therefore, preferably, the maximal amount of biomass for providing useful protein product is produced, but generally no more.

Thereafter, additional plants of the same variety containing the same expression system intended to express the same desired protein or proteins to yield, about the same quantity of desired protein are planted in the same or similar space. This can occur about 2, 3, 4, 5 or more times in a fixed period of months or years. After each planting/harvesting cycle, proteins of interest are separated from the biomass obtained to yield substantially pure proteins suitable for uses such as, for example, drugs. Thus, in contrast to the use of Arabidopsis for research purposes, identical plants (i.e., seeds from a stable transgenic line of plant expressing an optimal construct) are planted over and over again to obtain biomass and to isolate characterized protein product(s) from such plants. Preferably, seeds are produced rapidly (e.g., in less than about 8-10 weeks).

The unique morphology of Arabidopsis also permits efficient utilization of space to maximize the amount of biomass produced. Arabidopsis has a small compact growth morphology that gives rise to a rosette of leaves. Within about 5-8 weeks time the entire surface of a one square foot area at a seeding density of between 10-15 seeds/ft² can be completely covered by a dense mat of leaves which extend approximately 2-5 cm from the surface of the growth substrate. At this time there is a similar amount of biomass being produced in the form of roots. Because of the low growth stature of the plant at this stage, it is possible to vertically stack many shelves on top of one another to grow the plants (i.e., at least about 2, at least about 3, at least about 4, at least about 5, at least about 6, at least about 7, at least about 8, at least about 9, at least about 10). On the other hand, if it is necessary to increase seed supply, this is easily accomplished by growing the plants under more suitable light regimes and providing enough room for the flower bolt to emerge. In general, it takes from about 8-10 weeks to go from planted seed to next seed harvest and each plant produces at least hundreds of seed.

Generally, a 20′×20′ growth chamber in accordance with the present invention, as described above, will produce at least 0.1%, preferably at least 0.5% and more preferably 1% or greater of a desired protein based on the weight of the total soluble protein recovered by harvesting the Arabidopsis grown in the growth chamber in a single growth/harvest cycle.

Phrased in terms of another measure, from this single 20′×20′ growth chamber, preferably at least about 1 gm (e.g., 100 g/ft.²×400 ft²×6 layers×10 g protein/1000 g biomass×0.1% desired protein of total protein=2.4 gm) of the desired protein will be produced, more preferably, at least about 5 gm, and even more preferably, at least about 10 g of the desired protein of interest will be produced. More preferably, the protein will be produced in an amount of at least about 500 mg, 1 gm, 2.5 gm, 5 gm, 7 gm, 8 gm, 9 gm, or at least about 10 g of recombinant protein.

Production of these quantities of protein can be absolute, i.e., time independent. That is to say, a particular growth chamber can be used over and over again until the desired level of the intended protein has been produced. When expressed in these terms, it is not important whether, for example, 1 g is produced as a result of a single planting that year, which produces the desired protein in an amount that is greater than 1% of the total soluble protein recovered, or as a result of 8-10 planting/harvesting cycles, each occurring every 35-45 days, producing a far less concentrated amount of the intended protein over the course of roughly the same period of a year.

If Arabidopsis is grown under less than desirable conditions, this may alter the harvesting windows to some degree. For example, at temperatures above 25° C., harvest may begin at 35 days. At temperatures below 20° C., while leaf production might generally be favored, the overall plant will be stressed and relatively unproductive.

As previously noted, certain of the factors discussed above are scalable. For example, overall yield is a function of a number of factors, including, without limitation, the density to which the plants are planted, the extent to which growth is allowed to continue, the number of cycles of planting and harvesting that will occur in a given space over the course of a given period of time such as, for example, a year, the amount of protein expressed in a given plant, etc. But also, the extent of planting has a large role to play in the eventual yield of protein. The foregoing example considered a growth chamber having 20 feet×20 feet of growing area in a single layer of plantable surface. However, in general, in growth chambers or greenhouses, it is possible to stack two or more individual layers in a given space, such as in tiers or on multilayered carts. The yield would therefore be multiplied by the number of layers planted in a given space. Preferably, a growth chamber is provided with at least about two layers of plants, at least a portion of which is cultivated for biomass which is not seed.

Yield can be reported as a ratio of area in terms of square feet. For example, if 4 g of intended protein were produced in a 20′×20′ growth chamber having a single layer of growth medium over the course of a year, the yield that year could be expressed as 4 g per 400 sq ft per year. If planting was conducted over several acres, the yield should be, on average, about the same when considered on a 400 sq ft basis. The same measure could also be used if two layers were planted in the same growth chamber on the assumption that the total square footage planted was 800 sq ft and the total amount of protein realized as isolated from the total soluble protein was 8 g in the same year. The ratio would still be 4 g per 400 sq ft per year. The minimum and maximum area planted will be dictated by a number of factors such as available space, i.e., number of chambers, acres, etc., the practical yield of the variety and expression cassette system selected, the desired total quantity of protein necessary and the time constraints, if any. If more protein is necessary in a short period of time, then a greater surface area needs to be planted and/or more planting/harvesting cycles need to be used. Possibly, a more efficient expression system would need to be developed.

The minimum amount of space planted should be that which would provide at least about 100 mg of the desired protein in a year, more preferably at least about 300 mg of the desired protein in a year, even more preferably at least about 500 mg of the desired protein in a year, still more preferably at least about 700 mg of the desired protein in a year and most preferably at least 1 g or more of the desired protein in a year. The example given throughout this text (20′×20′ growth room) is intended as a reference point. All aspects of the process are scalable in terms of space and time to produce a certain amount of a specific product. Space and time aspects can be positively or negatively impacted based on the percent yield for any particular protein in any particular host strain of Arabidopsis.

Even at the scale of a 20′×20′ room, it is preferred that an automated or semi-automated process for harvesting the plant material be employed. Depending on the actual growth substrate (soil versus hydroponic), there are systems that would be preferred. Purification of proteins from massive amounts of fresh plant tissue can be accomplished by a number of methods some of which can be found in U.S. Pat. No. 6,096,546, W0 00009725, and W09946288 Protein Purification.

Arabidopsis is amenable to growth in a variety of culture room and greenhouse conditions. It is possible to modify the grow conditions such as intensity of light and day-length to favor production of leafy biomass versus conversion to floral development. In general, shorter day-lengths (8-10 hours) favor a more leafy phenotype while longer day-lengths (>12 hours) promote flowering and seed development. Growth temperature also impacts morphology and development with cooler temperatures favoring more leafy growth. Thus, in general, 8-10 hour day length and growth temperatures between 20° C.-23° C. will favor leafy vegetative growth compared to 12-14 hour day length and 24° C.-25° C., which will favor faster maturation and production of seed. While Arabidopsis is rather prolific in regards to seed multiplication rates, the seed is extremely small and is not the desired harvestable product for the protein. In this work the protein of interest is expressed and isolated from the vegetative portions of the plant (although it may also be expressed in the seed).

In one embodiment, plants are grown in 2-inch high flats in Metromix 350 for 35 days at 25° C. with a 10-hour day-length. At a seeding density of between 10-15 plants per square foot, one can readily generate 100-150 grams per square foot of total fresh weight. Approximately 1 gram of that is total soluble protein. Relative expression levels for any particular transgene product, levels of at least 0.1%-1% of total soluble protein are achieved. Preferably, at least about 1-5%, and more preferably, greater than 5% of the total soluble protein isolated as biomass is a desired recombinant protein. Milligram and preferably, up to gram quantities of pure protein are obtained from 100 square feet of Arabidopsis seedlings for the purpose of commercial large-scale production. While Arabidopsis is not very large in stature or appreciated for leaf biomass. This work demonstrates, that when used for high density growth, it can produce a very good total yield of biomass relative to the total volume of space, time, energy and inputs necessary to grow the plant.

The present invention identifies uses of the plant Arabidopsis thaliana for mass production of proteins, in particular, this includes proteins to be produced under conditions suitable for use in such regulated fields as pharmaceuticals and diagnostic reagents.

Isolation of Proteins

After cultivation, biomass is harvested to recover recombinant proteins. This harvesting step may comprise harvesting entire plants, or only the leaves, or roots or cells of the plant. This step may either kill the plant or, if only a portion of the transgenic plant is harvested, may allow the remainder of the plant to continue to grow. However, preferably, at least a portion of the entire biomass is in a growth zone (i.e., an area or a growth chamber such as a green house) is harvested which includes all plant tissue including seed. The remaining portion may be used to obtain seed for replanting and the plants from which seeds are collected may be allowed to continue to grow or can be added to the biomass collected to recover recombinant protein.

After harvesting, protein isolation may be performed using methods routine in the art. For example, at least a portion of the biomass may be homogenized, and recombinant protein extracted and further purified. Extraction may comprise soaking or immersing the homogenate in a suitable solvent. As discussed above, proteins may also be isolated from interstitial fluids of plants, for example, by vacuum infiltration methods, as described in U.S. Pat. No. 6,284,875.

Purification methods include, but are not limited to, immuno-affinity purification and purification procedures based on the specific size of a protein/protein complex, electrophoretic mobility, biological activity, and/or net charge of the recombinant protein to be isolated, or the presence of a tag molecule in the protein.

However, in one aspect, recombinant proteins are not isolated but fractions of the biomass are obtained for oral administration to an animal (e.g., such as a human being). Such fractions may be provided in forms which include, but are not limited to, tablets, capsules, pellets, and suspensions (e.g., in the form of drinks, syrups, etc.). In one aspect therefore, the method comprises orally administering to an animal Arabidopsis cells or fractions thereof.

Pharmaceutical Compositions

Recombinant proteins isolated from Arabidopsis can be used in methods of preventing or treating pathologies, for nutritional value, as a nutritional supplement, as a cosmetic, as an antimicrobial agent, for eliciting desired immune responses (e.g., as vaccines), and the like.

In one aspect of the invention, a recombinant protein or biologically active fragment thereof obtained from an Arabidopsis biomass, is formulated as a pharmaceutical composition. Preferably, a pharmaceutical composition is a sterile aqueous or non-aqueous solution, suspension or emulsion, which additionally comprises a physiologically acceptable carrier (i.e., a non-toxic material that does not interfere with the activity of the active ingredient). More preferably, the composition also is non-pyrogenic and free of viruses or other microorganisms. Any suitable carrier known to those of ordinary skill in the art may be used. Representative carriers include, but are not limited to: physiological saline solutions, gelatin, water, alcohols, natural or synthetic oils, saccharide solutions, glycols, injectable organic esters such as ethyl oleate or a combination of such materials. Optionally, a pharmaceutical composition additionally contains preservatives and/or other additives such as, for example, antimicrobial agents, anti-oxidants, chelating agents and/or inert gases, and/or other active ingredients.

Routes and frequency of administration, as well doses, will vary from patient to patient and according to the condition being prevented or treated or the benefit being conferred (e.g., where provided as a nutritional supplement). In general, pharmaceutical compositions are administered intravenously, intraperitoneally, intramuscularly, subcutaneously, topically, by inhalation, etc. However, the exact method of administration is non-limiting. A effective dose of recombinant protein or biologically active fragment thereof is administered.

As used herein, an effective dose is an amount that is sufficient to show improvement in the symptoms of a patient with a pathological condition or an amount sufficient to confer a benefit on a patient. Such improvement or benefit may be detected by monitoring appropriate clinical or biochemical endpoints as is known in the art. In general, the amount of recombinant protein present in a dose ranges from about 1 μg to about 100 mg per kg of host. Suitable dose sizes will vary with the size of the patient, but will typically range from about 10 mL to about 500 mL for 10-60 kg animal. A patient can be a mammal, such as a human, or a domestic animal.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated as being incorporated by reference herein.

Those skilled in the art will recognize, or be able to ascertain, using no more than routine experimentation, numerous equivalents to the specific substances and procedures described herein. Such equivalents are considered to be within the scope of this invention, and are covered by the following claims.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

We claim:

1. A method for producing large scale amounts of a recombinant protein in Arabidopsis, comprising:
(a) introducing at least one expression cassette capable of expressing the recombinant protein into Arabidopsis cells;
(b) identifying a cell which expresses a desired level and/or activity of the recombinant protein;
(c) obtaining Arabidopsis seeds from progeny of the cell;
(d) cultivating the seeds under conditions to produce seed rapidly; and
(e) screening plants obtained from the seeds to identify plants which express a desired level and/or activity of recombinant protein;
(f) cultivating at least two generations of the protein producing plants and selecting the highest protein producers under conditions to produce seeds rapidly; and
(g) cultivating a plant line expressing the highest amount of protein, under conditions to produce at least about 50 grams of biomass per square foot.

2. The method according to claim 1, wherein at least about 100 grams of biomass per square foot is produced.

3. The method according to claim 1, wherein at least about 200 grams of biomass per square foot are produced.

4. A method for producing a recombinant protein in Arabidopsis, comprising:
a. growing an Arabidopsis variety comprising at least one expression cassette for expressing a recombinant protein, under conditions that promote the production of vegetative and leafy biomass;
b. harvesting at least a portion of the Arabidopsis containing recombinant protein prior to seed formation; and
c. recovering at least one gram of recombinant protein in a one year period.

5. The method according to claim 1 or 4, wherein the Arabidopsis is preselected for maximal expression and/or activity of protein.

6. The method according to claim 1 or 4, wherein the Arabidopsis exhibits reduced levels of posttranslational glycosylation of proteins.

7. The method according to claim 6, wherein Arabidopsis comprises a human glysosylase transferase gene.

8. The method according to claim 7, wherein the Arabidopsis is a cgl or mur mutant.

9. The method according to claim 1, further comprising the step of preselecting an Arabidopsis strain which produces an increase in average biomass in comparison to wild type Arabidopsis strains and obtaining Arabidopsis cells from the preselected strain.

10. The method according to claim 1 or 4, wherein at least one expression cassette is introduced into Arabidopsis cells by infiltration.

11. The method according to claim 10, wherein infiltration is done under a vacuum.

12. The method according to claim 4, wherein a portion of the Arabidopsis is cultivated until seed formation and seeds are obtained from the portion.

13. The method according to claim 12, wherein at least some of the seeds are replanted.

14. The method according to claim 4, wherein steps (a)-(b) occur repetitively over at least a six-month period.

15. The method according to claim 1 or 4, wherein the expression construct comprises a gene expressing the recombinant protein operably linked to a regulatory sequence.

16. The method according to claim 15, wherein the regulatory sequence comprises one or more of a promoter, enhancer sequence, transcription terminator, or IRES element.

17. The method according to claim 15, wherein the recombinant protein is selected from the group consisting of: a growth factor, receptor, ligand, signaling molecule, kinase, tumor suppressor, blood clotting protein, cell cycle protein, telomerase, metabolic protein, enzyme, a protein deficient in a human patient with a pathological condition, an antibody, an antigen, insulin, albumin, an interferon, and a cytokine.

18. The method according to claim 1 or 4, wherein the expression cassette expresses a plurality of recombinant proteins.

19. The method according to claim 1 or 4 wherein the expression cassette expresses a polycistronic mRNA.

20. The method according to claim 19, wherein the expression cassette expresses a multi-subunit protein.

21. The method according to claim 20, wherein the multisubunit protein is selected from the group consisting of a T Cell Receptor, an MHC molecule, a protein of the immunoglobulin superfamily, a nucleic acid binding protein, a multi-subunit enzyme, and a multi-subunit abzyme.

22. The method according to claim 1 or 4, wherein the protein is a human protein.

23. The method according to claim 1 or 4, wherein the protein is a pharmaceutical agent, a diagnostic protein, a nutriceutical, a cosmeceutical, and a veterinary agent.

24. The method according to claim 1 or 4, wherein the protein is a fusion protein.

25. The method according to claim 24, wherein the fusion protein comprises an effector polypeptide.

26. The method according to claim 24, where the fusion protein comprises a transcriptional activating polypeptide which increases transcription of the fusion protein.

27. The method according to claim 24, wherein the fusion protein comprises a tag polypeptide.

28. The method according to claim 24, wherein the fusion protein comprises a linker polypeptide.

29. The method according to claim 28, where in the linker polypeptide is a cleavable linker.

30. The method according to claim 15, wherein the regulatory sequence comprises a promoter which is active in greater than 50% Arabidopsis plant tissue in a plant about 20-40 days old.

31. The method according to claim 15, wherein the regulatory sequence comprises a promoter which is active in at least one or more of: leaf, stem and root tissue.

32. The method according to claim 15, wherein the regulatory sequence is a promoter selected from the group consisting of Arabidopsis Actin 2 promoter, the OCS(MAS) promoter, the CaMV 35S promoter, the figwort mosaic virus 34S promoter, and a chloroplast promoter.

33. The method according to claim 1 or 4, wherein the protein comprises a targeting sequence.

34. The method according to claim 1 or 4, wherein the targeting sequence is capable of targeting the recombinant protein to a specific location in a plant cell selected from the group consisting of: the cell membrane, extracellular space, a plastid, and an endomembrane.

35. The method according to claim 34, wherein the targeting sequence is calreticulin or substilisin.

36. The method according to claim 24, wherein the fusion protein comprise a site-specific cleavage site.

37. The method according to claim 1 or 4, further comprising isolating the protein.

38. A biomass of Arabidopsis comprising at least about 10 grams, wherein at least 0.1% of the soluble protein of said Arabidopsis biomass comprises a recombinant protein.

39. The biomass according to claim 38, wherein the biomass comprises more than seed.

40. A method of providing a protein to a human being comprising orally administering Arabidopsis cells or a fraction thereof to the human being.

41. The method according to claim 40, wherein the protein is not naturally expressed in Arabidopsis.

42. The method according to claim 40, wherein the protein is encoded by a recombinant gene expressed in the Arabidopsis cells.

43. The method according to claim 40, wherein the cells comprise an antigen for eliciting an effective immune response.

44. The method according to claim 40, further comprising harvesting biomass from at least a portion of the Arabidopsis produced, wherein the biomass is not seed.

45. The method according to claim 44, wherein said harvesting occurs at least about 2 times over about two growth cycles.

46. The method according to claim 44, wherein said harvesting occurs at least about 5 times over about five growth cycles.

47. The method according to claim 44, wherein said harvesting occurs at least about 10 times over about ten growth cycles.

48. The method according to claim 44, wherein said harvesting occurs at least about 2 times over about more than two growth cycles.

49. The method according to claim 44, wherein said harvesting occurs at least about 5 times over about more than five growth cycles.

50. The method according to claim 45 or 46, wherein there is at least one growth cycles when biomass is not harvested.
